(19) United States
(12) Patent Application Publication
(10) Pub. No.: US 2026/0091186 A1
(43) Pub. Date: Apr. 2, 2026

(54) MEDICAL GAS DELIVERY

(71) Applicant: SLEEP EASY TECHNOLOGY INC., South Bend, IN (US)

(72) Inventors: Anthony ESPLIN, South Bend, IN (US); Aaron ESPLIN, South Bend, IN (US)

(21) Appl. No.: 19/344,352

(22) Filed: Sep. 29, 2025

Related U.S. Application Data
(63) Continuation of application No. PCT/US2024/021801, filed on Mar. 27, 2024.
(60) Provisional application No. 63/454,756, filed on Mar. 27, 2023.
(60) Provisional application No. 63/454,993, filed on Mar. 28, 2023.

(51) Int. Cl. A61M 16/00; A47G 9/02; A61F 5/37

(52) U.S. Cl. CPC A61M 16/0003; A47G 9/0253; A61F 5/3707

(57) ABSTRACT

A system for delivering medical gas, such as supplemental oxygen, to a subject includes a positioner and a diffuser. The positioner includes a positioning wall with an aperture that receives and may couple to a proximal portion of the diffuser. The system may also include a pillowcase with one or more fasteners that hold the positioner and a diffuser coupled thereto at a desired location and orientation over a pillow where a subject who is to receive medical gas will place their head. The pillowcase may receive two positioners and associated diffusers. Methods of administering medical gas and methods for maintaining medical gas delivery systems are also disclosed.

### CROSS-REFERENCE TO RELATED APPLICATIONS

This application is a continuation of International application no. PCT/US2024/021801, titled MEDICAL GAS DELIVERY and filed on Jun. 12, 2024 (“the '801 PCT Application”), which claims for priority to the Mar. 27, 2023 filing date of U.S. Provisional Patent Application 63/454,756, titled MEDICAL GAS DELIVERY (“the '756 Provisional Application”) and the Mar. 28, 2023 filing date of U.S. Provisional Patent Application No. 63/454,993, titled MEDICAL GAS DELIVERY (“the '993 Provisional Application”). The entire disclosures of the '801 PCT Application, the '756 Provisional Application, and the '993 Provisional Application are hereby incorporated herein.

### TECHNICAL FIELD

This disclosure relates to devices for delivering medical gases, such as supplemental oxygen, to a subject. More specifically, this disclosure relates to devices that deliver medical gases to individuals as they lie in bed. Such a device positions a diffuser in a direction that enables the diffuser to deliver medical gas to the face of the subject. Methods for delivering medical gases are also disclosed.

### RELATED ART

A variety of health conditions may lead to the need for the delivery of medical gases, such as supplemental oxygen. Among other conditions, supplemental oxygen helps subjects who suffer from certain types of sleep apnea, acute bronchitis, asthma, heart conditions, chronic obstructive pulmonary disease (COPD), dementia, Alzheimer's disease, environmental needs such as high altitude sickness, and other disorders to achieve more stable, restful states of sleep than those subjects typically experience without supplemental oxygen. A variety of apparatuses, including conventional oxygen delivery systems, may be used to provide home oxygen therapy patients with supplemental oxygen as they sleep.

A conventional oxygen delivery system typically includes an oxygen source (e.g., an oxygen concentrator, an oxygen tank, liquid oxygen, etc.), tubing, and a nasal cannula that delivers oxygen from the oxygen concentrator to a subject's nose. An oxygen concentrator concentrates oxygen from the air, providing air with an above-ambient concentration of oxygen, which is also referred to herein as “supplemental oxygen” for the sake of simplicity. The oxygen source delivers the supplemental oxygen to and through tubing to the nasal cannula. The nasal cannula has a configuration that enables it to be secured beneath a subject's nose in an orientation that directs the consistent flow of gas, such as supplemental oxygen, into the subject's nose. It is up to the patient to breathe in to access that oxygen from the nasal cannula. If the patient is a mouth breather, as over 70% of the general population are when they sleep, the concentrated oxygen is not inhaled. Nasal cannulas may also be ineffective for delivering supplemental oxygen to subjects who suffer from upper respiratory tract infections (e.g., a common cold, influenza, sinus infections, etc.) and to subjects who suffer from nasal abnormalities (e.g., a deviated septum, etc.).

The use of a nasal cannula can be uncomfortable and irritating. The presence of the nasal cannula beneath the subject's nose may be bothersome (e.g., it may lead to nose bleeds when the air is dry, such as during wintertime, etc.). In addition, the delivery of supplemental oxygen into a subject's nose has a tendency to dry out the subject's nasal membranes, which may make the nasal cannula even more bothersome. The discomfort a subject experiences while wearing a nasal cannula and while the nasal cannula delivers regulated, supplemental oxygen to the subject's nose may lead to removal of the nasal cannula-either intentionally or unintentionally—as the subject tries to sleep. When the nasal cannula is removed or the subject breathes through their mouth, however, the subject no longer receives supplemental oxygen, which may cause the same problem the supplemental oxygen is intended to address (e.g., fitful, interrupted sleep for hypoxia patients, etc.).

### SUMMARY

An embodiment of a medical gas delivery system of this disclosure may include a positioner and a diffuser. The diffuser may also be referred to as a nozzle. In some embodiments, the medical gas delivery system may include a pair of positioners and diffusers that are spaced apart and oriented to oppose each other so that they may deliver medical gas to a patient's face regardless of the orientation of their face (e.g., upward, to the left, to the right, etc.). For example, the positioners may be placed on opposite sides of a pillow. Optionally, the medical gas delivery system may include a cover, such as a pillowcase.

The positioner may include a receptacle for the diffuser. The receptacle may be at least partially defined by a positioning wall. More specifically, a concave front surface of the positioning wall may define a recess that, in turn, forms part of the receptacle. The positioning wall may be curved to impart the front surface of the positioning wall with the concave shape that defines the recess. Alternatively, the positioning wall may be defined by a plurality of sections that impart the front surface of the positioning wall with the concave shape; thus, the front surface of the positioning wall may define the recess.

The positioning wall may have a configuration that enables it to couple to the diffuser. In some embodiments, the positioning wall may include an aperture that is shaped complementary to and receives a proximal portion of the diffuser. The aperture may include a coupler that engages the proximal portion of the diffuser (e.g., in a snap-fit arrangement, in a threaded arrangement, etc.).

A ledge, or an awning, may be positioned on top of the positioning wall. The ledge may extend over or even beyond the recessed area defined by the concave front surface of the positioning wall. A front edge of the ledge may have a convex shape. The front edge may be curved. In embodiments where the positioning wall is also curved, the curvatures of the positioning wall and the front edge of the ledge may be the same, but oppositely oriented.

Dimensions of the positioning wall (e.g., its height, the depth of the recess it defines, etc.) and the ledge, if any, may provide for a receptacle that can receive an entirety of a diffuser.

Optionally, the positioner may include one or more support walls behind the positioning wall of the positioner. Opposite outer edges of the one or more support walls may be joined to outer edges of the positioning wall. At least portions of the one or more support walls may be spaced apart from the positioning wall. Together, the positioning wall and the one or more support walls may stabilize the positioner as it is placed in an upright position on a surface (e.g., a pillow, a mattress, etc.).

The diffuser includes the proximal portion, as well as an intermediate portion and a distal portion, or a head. When the diffuser is assembled with a positioner, it may be placed in a receptacle defined by the positioning wall and the ledge of the positioner. Alternatively, the diffuser may be placed within a recess defined by the positioning wall of a positioner, while the diffuser may include a ledge that may be positioned adjacent to a top of the positioning wall.

The proximal portion of the diffuser may couple the diffuser to the positioning wall of the positioner or, more specifically, to the coupler and aperture of the positioning wall. In some embodiments, a configuration of the proximal portion of the diffuser may enable a proximal end of the diffuser to be positioned beyond (behind) the positioning wall of the positioner.

In addition to coupling to the positioning wall of the positioner, the proximal portion of the diffuser couples to a conduit (e.g., a tube, etc.) and includes an inlet (e.g., a proximal opening, a proximal conduit, etc., at its proximal end) to enable the diffuser to receive a medical gas from a source of the medical gas.

Optionally, the proximal portion of the diffuser (e.g., an inlet of the diffuser, etc.) may increase turbulence in gases that are delivered by the diffuser, which may facilitate the creation of a “gas cloud” for inhalation by a subject. Such an inlet may include a baffle carried by the proximal portion of the diffuser (e.g., over a channel and/or an aperture the inlet, etc.). More specifically, the baffle may be positioned within a flow path defined by the inlet. A configuration (e.g., a shape, dimensions, etc.) and a position of the baffle may cause gas that flows into the inlet to flow around the baffle and then toward and through the distal portion of the diffuser, albeit less forcefully and more diffusely than if the gas were to flow unimpeded through the inlet. In some embodiments, the baffle may be removable from the inlet and replaceable within the inlet.

A distance across the diffuser (e.g., its diameter) increases along an intermediate portion of the diffuser. A distal portion of the diffuser may be even more enlarged, with a distal end of the diffuser comprising an opening, or a delivery face, through which the medical gas is expelled. A shape of the diffuser, including its distal portion, may enable the diffuser to deliver the medical gas in a somewhat controlled manner. The distal portion of the diffuser may have a size and a shape that enable gas to be delivered efficiently and effectively to the face of a subject. Use of the diffuser includes providing medical gas flow from its source, through the conduit, through the proximal and intermediate portions of the diffuser, into the distal portion of the diffuser, and out of the delivery face.

In some embodiments, the diffuser may include a useful life indicator that may provide a readily perceptible indication that the diffuser should be replaced, or when a useful life of the diffuser has ended. Such an indicator may be located on or adjacent to the delivery face of the diffuser. Without limitation, the indicator may comprise a material that changes color. The material may change color over a predetermined duration of time, after use of the diffuser for a predetermined amount of time, when exposed to a predetermined amount of the medical gas, or the like.

In another aspect, a cover, such as a pillowcase, of a medical gas delivery system is disclosed. The pillowcase may receive and place the positioner and, thus, the diffuser in a desired orientation on a pillow. The pillowcase may include an opening and a receptacle that enable it to receive a pillow. The pillowcase may also include a positioner fastener. The positioner fastener may hold the positioner in place in a desired orientation (i.e., with the positioning wall positioning the delivery face of the diffuser in a desired orientation) toward a more central location on the upper surface of the pillowcase. The positioner fastener may position the delivery face of the diffuser a suitable distance from the head of a subject.

In some embodiments, a pillowcase may include two or more positioner fasteners. As non-limiting examples, a positioner fastener may be provided adjacent to each side edge of the pillowcase, positioner fasteners may be provided at different corners of the pillowcase, etc. Each positioner fastener may receive a positioner and hold it in place in a desired orientation (i.e., with the positioning wall positioning the delivery face of the diffuser in a desired orientation) toward a more central location on the upper surface of the pillowcase.

The positioner fastener may engage and hold the positioner in any of a variety of desired orientations. More specifically, the positioner fastener may engage a complementary positioner fastener on the positioner. In a specific embodiment, the positioner fastener may comprise a touch fastener (e.g., a Velcro fastener, etc.), which may engage a complementary touch fastener on a bottom of the positioner. Such a touch fastener may comprise a strip secured to the upper surface of the pillowcase or a fabric (e.g., a loop fabric, etc.) of at least a portion of the upper surface of the pillowcase.

Optionally, the pillowcase may include a fabric cover (e.g., a lip, a flap, etc.) that may be positioned to cover each positioner fastener. The fabric cover may comprise a positioner receptacle. Each positioner receptacle may cover or be positioned to cover at least one positioner fastener of the pillowcase. Each positioner receptacle may be provided on an upper surface of the pillowcase, adjacent to a side edge of the pillowcase. In some embodiments, a pillowcase may include two or more positioner receptacles. As non-limiting examples, a positioner receptacle may be provided adjacent to each side edge of the pillowcase, positioner receptacles may be provided at different corners of the pillowcase, etc. Each positioner receptacle may receive a positioner and enable it to be held in place (e.g., by a positioner fastener, etc.) in a desired orientation (i.e., with the positioning wall positioning the delivery face of the diffuser in a desired orientation) toward a more central location on the upper surface of the pillowcase.

An outer side of each positioner receptacle may include a conduit receptacle that may comprise a small opening adjacent to the side edge of the pillowcase. Such a conduit receptacle may receive a conduit, enabling a distal end of the conduit to be positioned within an interior of the positioner receptacle. Such a conduit may convey medical gas from its source to a diffuser carried by a positioner located within the interior of the positioner receptacle.

Each positioner receptacle includes a primary opening that may face a remainder (e.g., more central locations, etc.) of the upper surface of the pillowcase. The primary opening enables the positioner receptacle to receive a positioner and a diffuser coupled with the positioner. In addition, the primary opening may enable gas to flow unimpeded from the delivery face of the diffuser to a location over the upper surface of the pillowcase (e.g., where an individual's nose and/or mouth are located, etc.).

Each positioner receptacle may have a shape and/or include features that enable it to at least partially engage the positioner. For example, an edge of the positioner receptacle that defines the primary opening may comprise an elastic element that, when relaxed, minimizes a size of the primary opening and, when expanded, provides access to the interior of the positioner receptacle.

Each positioner receptacle may comprise an elongated element that enables a positioner to be positioned at a plurality of different locations along the side edge of the pillowcase. In some embodiments, an elongated positioner receptacle may be used in conjunction with an elongated positioner fastener. An elongated positioner receptacle may enable the positioner to be placed on the pillowcase in such a way that the delivery face of the diffuser may be oriented at a variety of different angles across the upper surface of the pillowcase. Without limitation, an elongated positioner receptacle may hold a positioner near a bottom edge of the pillowcase and orient the positioner at an angle that will enable the delivery face of the diffuser to deliver medical gas at a somewhat upward angle (relative to the bottom edge of the pillowcase) toward a subject's nose and mouth. An elongated positioner receptacle may also hold at positioner at an intermediate location along the length of the side edge of the pillowcase at an angle that will enable the delivery face of the diffuser to deliver medical gas horizontally across the upper surface of the pillowcase. In addition, an elongated positioner receptacle may hold a positioner near a top edge of the pillowcase and orient the positioner at an angle that will enable the delivery face of the diffuser to deliver medical gas at a somewhat downward angle (relative to the top edge of the pillowcase) toward the subject's mouth and nose.

In use, the edge of the positioner receptacle may be tucked between a lower surface of the ledge of the positioner and an upper extent of a diffuser that has been coupled to the positioner. A positioner receptacle may cover a positioner in a manner that prevents the subject from unintentionally contacting the positioner (e.g., as the individual moves as he or she sleeps or otherwise rests, etc.), which may minimize the possibility of the subject being injured by unintended contact with the positioner.

In some embodiments, a medical gas delivery system may include a pillowcase with at least one positioner receptacle, at least one positioner, and a diffuser that may be coupled to each positioner.

A medical gas may be delivered to a subject with such a medical gas delivery system by arranging the positioner on a positioner fastener and, optionally, within a positioner receptacle of a pillowcase on a pillow where the subject will rest their head. The positioner is placed on the positioner fastener and, optionally, within the positioner receptacle at a location and at an angle that will enable the delivery face of a diffuser coupled to the positioner to direct the medical gas to a location over the upper surface of the pillowcase where the subject's head will rest. In some embodiments, another positioner and diffuser may be arranged relative to another positioner fastener and, optionally, within another positioner receptacle on an opposite side of the pillowcase. Such an arrangement may ensure that the subject will receive the medical gas regardless of the orientation of their head on the pillow.

The subject's head may be placed on the upper surface of the pillowcase and, thus, on the pillow adjacent to the positioner(s). If desired, the position of each positioner may be adjusted (e.g., moved, re-oriented, etc.) to increase the subject's comfort, optimize the efficiency with which the medical gas delivery system delivers the gas to the subject, or the like. With the subject's head positioned on the surface, the medical gas may be delivered to and through the diffuser to the face of the subject.

Maintenance of the medical gas delivery system may include removal and disposal of the diffuser after a predetermined period of time, a predetermined number of uses, etc. The conduit between the source of medical gas and the diffuser should be replaced periodically. In addition, the positioner should be cleaned or sanitized periodically and the pillowcase, if any, should be laundered periodically. The amount of time for each of these actions may differ, depending upon exposure of a particular component to the subject. Alternatively, some or all of these actions may be performed on the same schedule (e.g., the pillowcase may be laundered, the diffuser replaced, and the positioner cleaned at the same time, etc., e.g., more frequently than the conduit is replaced).

Other aspects of the disclosed subject matter, as well as features and advantages of various aspects of the disclosed subject matter, should be apparent to those of ordinary skill in the art through consideration of the ensuing description, the accompanying drawings, and the appended claims.

### BRIEF DESCRIPTION OF THE DRAWINGS

In the drawings:

FIG. 1 is a front view of an embodiment of a positioner of this disclosure;

FIG. 2 is a bottom view of the embodiment of the positioner shown in FIG. 1;

FIG. 3 is a top view of the embodiment of the positioner shown in FIG. 1;

FIG. 4 is a side view of an embodiment of a diffuser of this disclosure;

FIG. 5 is a proximal end view of the embodiment of the diffuser shown in FIG. 4;

FIG. 6 is a perspective view showing a distal end of the embodiment of the diffuser shown in FIG. 4;

FIG. 7 is a top view of an embodiment of a pillowcase of this disclosure, showing an upper surface of the pillowcase and two positioner receptacles on the upper surface of the pillowcase; the measurements provided in FIG. 7 are specific to a pillowcase for a standard queen size pillow (i.e. 20 inches (about 25 cm) by 30 inches (about 38 cm);

FIG. 8 illustrates an upper surface of a partially disassembled pillowcase (the positioner receptacle having been partially pulled back or removed), showing a positioner fastener on the upper surface;

FIG. 9 is a bottom view of an embodiment of a medical gas delivery system including the embodiment of the positioner shown in FIG. 1 and the embodiment of the diffuser shown in FIG. 4 in an assembled relationship;

FIG. 10 is a top view of an embodiment of a medical gas delivery system including the embodiment of the pillowcase shown in FIG. 8 and the assembled positioner and diffuser shown in FIG. 9 positioned on and secured to the positioner fastener of the pillowcase;

FIG. 11 is a perspective view of the embodiment of the medical gas delivery system shown in FIG. 10;

FIGS. 12 and 13 are side view of an embodiment of the medical gas delivery system with a pillowcase that includes a fabric cover, or a positioner receptacle, showing the positioner receptacle covering the assembled positioner and diffuser and a delivery face of the diffuser exposed through the primary opening of the positioner receptacle; and

FIG. 14 shows an embodiment of use of a medical gas delivery system that includes a pair of positioners and diffusers secured to a pillow in a manner that enables an individual to rest their head on the pillow and to receive medical gas from one or both of the diffusers.

### DETAILED DESCRIPTION

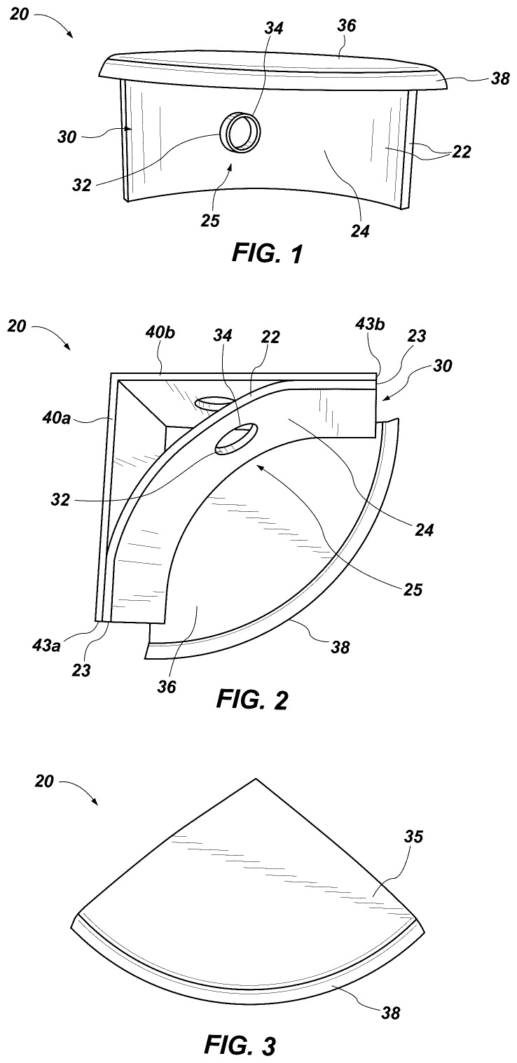

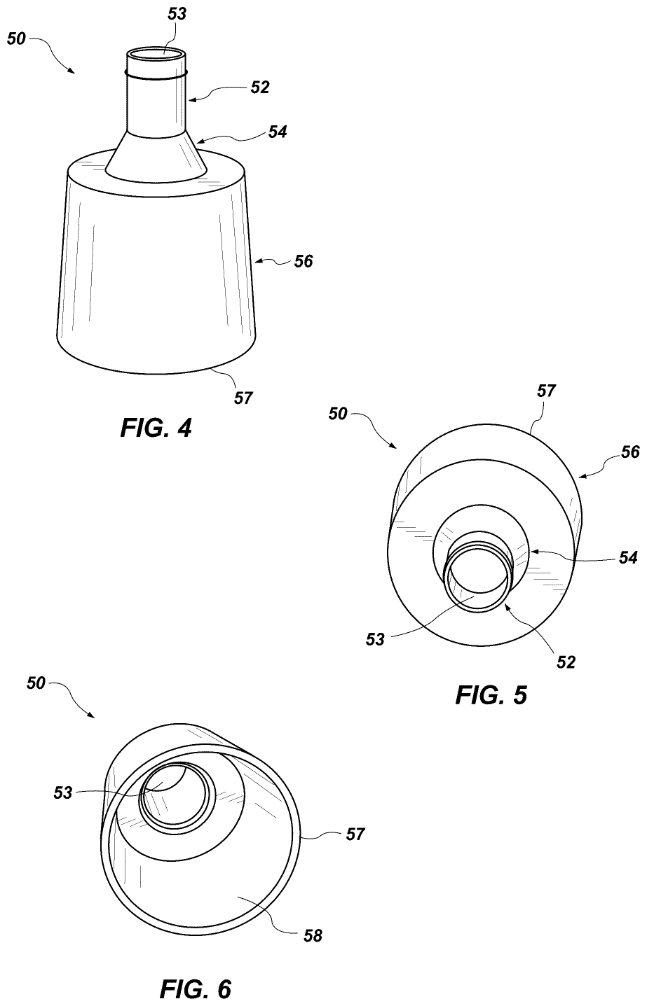

A medical gas delivery system 10 of this disclosure may include a positioner 20, an embodiment of which is depicted by FIGS. 1-3, and a diffuser 50, an embodiment of which is depicted by FIGS. 4-6.

With reference to FIGS. 1-3, the positioner 20 may include a receptacle 30 for the diffuser 50 (FIGS. 4-6). The receptacle 30 may be at least partially defined by a positioning wall 22 of the positioner 20. More specifically, a concave front surface 24 of the positioning wall 22 may define a recess 25 that forms part of the receptacle 30. The positioning wall 22 may be curved to impart the front surface 24 with the concave shape and, thus, to define the recess 25. Alternatively, the positioning wall 22 may be defined by a plurality of sections that impart the front surface 24 with the concave shape that defines the recess 25.

The positioning wall 22 may have a configuration that enables it to couple to the diffuser 50 (FIGS. 4-6). In some embodiments, the positioning wall 22 may include an aperture 32 that is shaped complementary to and receives a proximal portion 52 (FIGS. 4-6) of the diffuser 50. The aperture 32 may include a coupler 34 that engages the proximal portion 52 of the diffuser 50 (e.g., in a snap-fit arrangement, in a threaded arrangement, etc.).

The positioner 20 may include a ledge 36, or an awning, atop the positioning wall 22. The ledge 36 may extend over or even beyond the receptacle 30 defined by the concave front surface 24 of the positioning wall 22. A front edge 38 of the ledge 36 may have a convex shape. The front edge 38 may be curved. In embodiments where the positioning wall 22 is also curved, the curvatures of the positioning wall 22 and the front edge 38 of the ledge 36 may be the same, but oppositely oriented.

Dimensions of the positioning wall 22 (e.g., its height, the depth of the recess it defines, etc.) and the ledge 36 may provide for a receptacle 30 that can receive an entirety of a diffuser 50 (FIGS. 4-6). In some embodiments, the receptacle 30 may be able to accommodate a diffuser 50 with a diameter of at least 2 inches (5 cm).

Optionally, the positioner 20 may include one or more support walls 40a, 40b behind the positioning wall 22 of the positioner 20. Opposite outer edges 43a, 43b of the one or more support walls 40a, 40b may be joined to outer edges 23 of the positioning wall 22. At least portions of the one or more support walls 40a, 40b may be spaced apart from the positioning wall 22. Together, the positioning wall 22 and the one or more support walls 40a, 40b may stabilize the positioner 20 as it is placed in an upright position on a surface (e.g., a pillow, a mattress, etc.).

As shown in FIGS. 4-6, the diffuser 50 includes the proximal portion 52, as well as an intermediate portion 54 and a distal portion 56, or a head. The diffuser 50 may be placed in the receptacle 30 (FIGS. 1-3) of the positioner 20 (FIGS. 1-3).

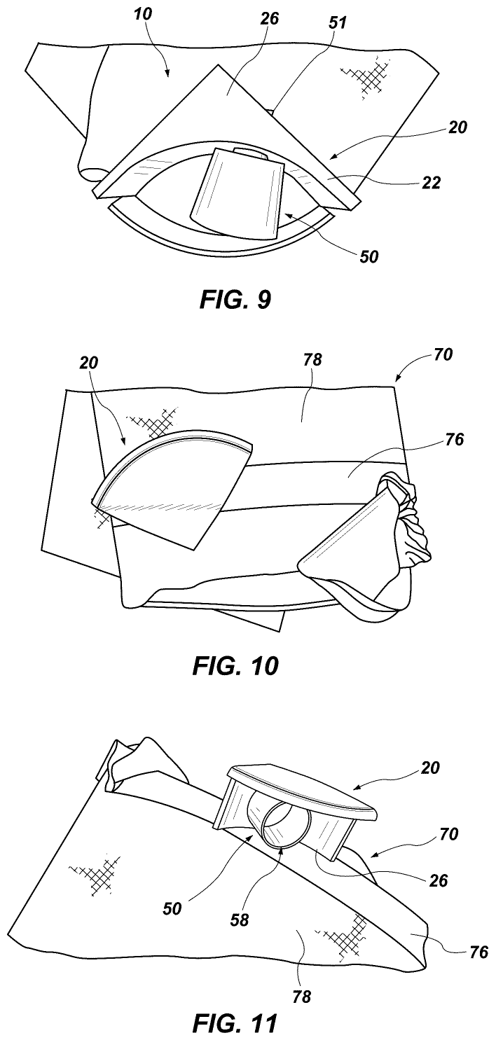

With added reference to FIG. 9, the proximal portion 52 of the diffuser 50 couples the diffuser 50 to the positioning wall 22 of the positioner 20 or, more specifically, to the coupler 34 and aperture 32 of the positioning wall 22. In some embodiments, a configuration of the proximal portion 52 may enable a proximal end 51 of the diffuser 50 to be positioned beyond (behind) the positioning wall 22 of the positioner 20.

The proximal portion 52 may also couple to a conduit (e.g., a tube, etc.) (not shown). The proximal portion 52 includes an inlet 53 (e.g., a proximal opening, a proximal conduit, etc., at its proximal end) to enable the diffuser 50 to receive a medical gas from a source of the medical gas (not shown).

Optionally, the proximal portion 52 of the diffuser 50 (e.g., the inlet 53 of the diffuser, etc.) may increase turbulence in gases that are delivered by the diffuser 50, which may facilitate the creation of a “gas cloud” for inhalation by a subject. Such an inlet 53 may include a baffle carried by the proximal portion 52 (e.g., over a channel and/or an aperture the inlet 53, etc.). More specifically, the baffle may be positioned within a flow path defined by the inlet 53. A configuration (e.g., a shape, dimensions, etc.) and a position of the baffle may cause gas that flows into the inlet 53 to flow around the baffle and then toward and through the distal portion 56 of the diffuser 50, albeit less forcefully and more diffusely than if the gas were to flow unimpeded through the inlet 53. In some embodiments, the baffle may be removable from the inlet 53 and replaceable within the inlet 53.

A distance across the diffuser 50 (e.g., its diameter) may increase along the intermediate portion 54 of the diffuser 50. The distal portion 56 of the diffuser 50 may be even more enlarged, with a distal end 57 of the diffuser 50 comprising an opening 58, or a delivery face, through which the medical gas is expelled. A shape of the diffuser 50, including its distal portion 56, may enable the diffuser 50 to deliver the medical gas in a somewhat controlled manner. The distal portion 56 may have a size (e.g., a diameter of about 0.5 inch to about 4 inches, etc.) and a shape (e.g., a cylindrical shape, or a cup shape, etc.) that enable gas to be delivered efficiently and effectively to the face of a subject. Use of the diffuser 50 includes providing medical gas flow from its source (not shown), through the conduit (not shown), through the proximal portion 52 and the intermediate portion 54 of the diffuser 50, into the distal portion 56 of the diffuser 50, and out of the opening 58.

In some embodiments, the diffuser 50 may include a useful life indicator 60 that may provide a readily perceptible indication that the diffuser 50 should be replaced, or a readily perceptible indication of when a useful life of the diffuser 50 has ended. Such an indicator 60 may be located on or adjacent to the opening 58 of the diffuser 50. Without limitation, the indicator 60 may comprise a material that changes color. The material may change color over a predetermined duration of time, after use of the diffuser 50 for a predetermined amount of time, when exposed to a predetermined amount of the medical gas, or the like.

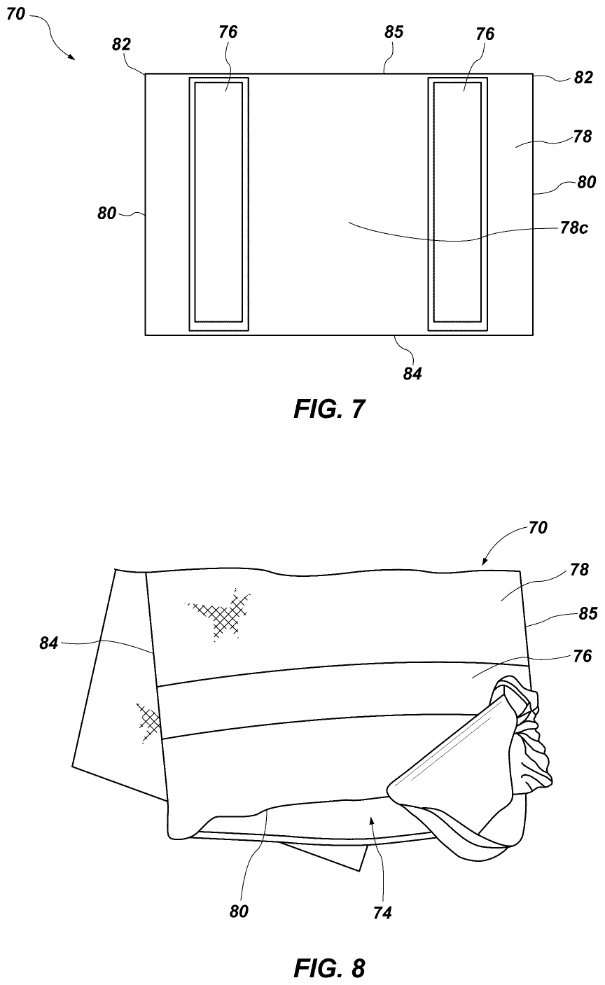

Turning now to FIGS. 7 and 8, an embodiment of a pillowcase 70 of a medical gas delivery system 10 is shown. FIG. 7 provides dimensions in inches. The pillowcase 70 may receive and place the positioner 20 (FIGS. 1-3) and, thus, the diffuser 50 (FIGS. 4-6) in desired orientations on a pillow (not shown). The pillowcase 70 may include an opening 72 and a receptacle 74 that enable the pillowcase 70 to receive the pillow. The pillowcase 70 may also include a positioner fastener 76. As shown in FIGS. 10 and 11, the positioner fastener 76 may hold the positioner 20 in place in a desired orientation (i.e., with the positioning wall 22 positioning the opening 58 of the diffuser 50 in a desired orientation) toward a more central location 78c on an upper surface 78 of the pillowcase 70. The positioner fastener 76 may position the opening 58 of the diffuser 50 eight inches (28.3 cm) or within eight inches (28.3 cm) from the head (e.g., face, nose, mouth, etc.) of a subject, five inches (12.7 cm) or within five inches (12.7 cm) from the head (e.g., face, nose, mouth, etc.) of a subject, between 0 inches (0 cm) and eight inches (28.3 cm) from the head of the subject, etc.

With continued reference to FIGS. 7 and 8, in some embodiments, the pillowcase 70 may include two or more positioner fasteners 76. As non-limiting examples, each positioner fastener 76 may be provided adjacent to each side edge 80 of the pillowcase 70 (as illustrated), positioner fasteners 76 may be provided at different corners 82 of the pillowcase 70, etc. Each positioner fastener 76 may receive a positioner 20 (FIGS. 1-3) and hold it in place in a desired orientation (i.e., with the positioning wall 22 (FIGS. 1-3) positioning the opening 58 (FIGS. 4-6) of the diffuser 50 (FIGS. 4-6) in a desired orientation) toward the central location 78c on the upper surface 78 of the pillowcase 70.

Referring again to FIGS. 10 and 11, each positioner fastener 76 may engage and hold a positioner 20 in any of a variety of desired orientations. More specifically, each positioner fastener 76 may engage a complementary positioner fastener 26 on the positioner 20. In a specific embodiment, the positioner fastener 76 may comprise a touch fastener (e.g., a Velcro fastener, etc.), which may engage the complementary positioner fastener 26-a complementary touch fastener-on a bottom 23 of the positioner 20. Such a positioner fastener 76 may comprise a strip secured to the upper surface 78 of the pillowcase 70 or a fabric (e.g., a loop fabric, etc.) of at least a portion of the upper surface 78 of the pillowcase 70. Without limitation, a positioner fastener 76 may have a length of about 0.5 inch (about 0.125 cm) to about 24 inches (about 60 cm) or more.

As shown in FIG. 7, a pillowcase 70 may include two positioner fasteners 76 on its upper surface 78. Each positioner fastener 76 may be positioned adjacent to a side edge 80 of the pillowcase 70. The positioner fasteners 76 may be spaced about 14 inches (about 35 cm) or more apart from each other.

Each positioner fastener 76 may enable a positioner 20 to be positioned at a plurality of different locations adjacent to the side edge 80 of the pillowcase 70. An elongated positioner fastener 76 may enable the positioner 20 to be placed on the pillowcase 70 in such a way that the opening 58) of the diffuser 50 may be oriented at a variety of different angles across the upper surface 78 of the pillowcase 70. Without limitation, an elongated positioner fastener 76 may hold a positioner 20 near a bottom edge 84 of the pillowcase 70 and orient the positioner 20 at an angle that will enable the opening 58 of the diffuser 50 to deliver medical gas at a somewhat upward angle relative to the bottom edge 84 of the pillowcase 70, toward a subject's nose and mouth. An elongated positioner fastener 76 may also hold at positioner 20 at an intermediate location along the length of the side edge 80 of the pillowcase 70 at an angle that will enable the opening 58 of the diffuser 50 to deliver medical gas horizontally across the upper surface 78 of the pillowcase 70. In addition, an elongated positioner fastener 76 may hold a positioner 20 near a top edge 85 of the pillowcase 70 and orient the positioner 20 at an angle that will enable the opening 58 of the diffuser 50 to deliver medical gas at a somewhat downward angle relative to the top edge 85 of the pillowcase 70, toward the subject's mouth and nose.

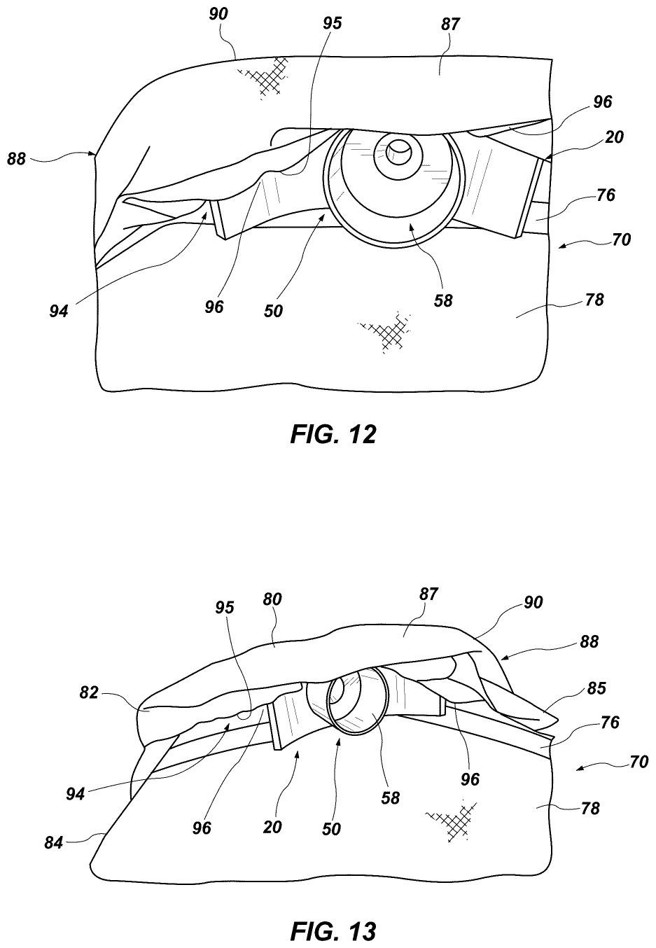

Optionally, as can be best seen in FIGS. 12 and 13, the pillowcase 70 may include a fabric cover 87 (e.g., a lip, a flap, etc.) that covers or may be placed over the positioner fastener 76 and any positioner 20 on the positioner fastener 76. The fabric cover 87 and the upper surface 78 of the pillowcase may define a positioner receptacle 88. Each positioner receptacle 88 may be provided on the upper surface 78 of the pillowcase 70, adjacent to a side edge of the pillowcase 80. In some embodiments, a pillowcase 70 may include two or more positioner receptacles 88. As non-limiting examples, a positioner receptacle 88 may be provided adjacent to each side edge 80 of the pillowcase 70, positioner receptacles 88 may be provided at different corners 82 of the pillowcase 70, etc. Each positioner receptacle 88 may receive a positioner 20 and enable it to be held in place (e.g., by a positioner fastener 76, etc.) in a desired orientation (i.e., with the positioning wall positioning the opening 58 of the diffuser 50 in a desired orientation) toward a more central location 78c on the upper surface 78 of the pillowcase 70.

An outer side 90 of each positioner receptacle 88 may include a conduit receptacle (not shown) that may comprise a small opening adjacent to the side edge 80 of the pillowcase 70. Such a conduit receptacle may receive a conduit (not shown), enabling a distal end of the conduit to be positioned within an interior 94 of the positioner receptacle 88. Such a conduit may convey medical gas from its source (not shown) to a diffuser 50 carried by a positioner 20 located within the interior 94 of the positioner receptacle 88.

Each positioner receptacle 88 includes a primary opening 96 that may face a remainder (e.g., more central locations 78c, etc.) of the upper surface 78 of the pillowcase 70. The primary opening 96 enables the positioner receptacle 88 to receive a positioner 20 and a diffuser 50 coupled with the positioner 20. In addition, the primary opening 96 may enable gas to flow unimpeded from the opening 58 of the diffuser 50 to a location over the upper surface 78 of the pillowcase 70 (e.g., where an individual's nose and/or mouth are located, etc.).

Each positioner receptacle 88 may have a shape and/or include features that enable it to at least partially engage the positioner 20. For example, an edge 95 of the positioner receptacle 88 that defines the primary opening 96 may comprise an elastic element that, when relaxed, minimizes a size of the primary opening 96 and, when expanded, provides access to the interior 94 of the positioner receptacle 88.

Each positioner receptacle 88 may comprise an elongated element that enables a positioner 20 to be positioned at a plurality of different locations along the side edge 80 of the pillowcase 70. In some embodiments, an elongated positioner receptacle may be used in conjunction with an elongated positioner fastener. An elongated positioner receptacle 88 may enable the positioner 20 to be placed on the pillowcase 70 in such a way that the opening 58 of the diffuser 50 may be oriented at a variety of different angles across the upper surface 78 of the pillowcase 70. Without limitation, an elongated positioner receptacle 88 may hold a positioner 20 near a bottom edge 84 of the pillowcase 70 and orient the positioner 20 at an angle that will enable the opening 58 of the diffuser 50 to deliver medical gas at a somewhat upward angle (relative to the bottom edge 84 of the pillowcase 70) toward a subject's nose and mouth. An elongated positioner receptacle 88 may also hold at positioner 20 at an intermediate location along the length of the side edge 80 of the pillowcase 70 at an angle that will enable the opening 58 of the diffuser 50 to deliver medical gas horizontally across the upper surface 78 of the pillowcase 70. In addition, an elongated positioner receptacle 88 may hold a positioner 20 near a top edge 85 of the pillowcase 70 and orient the positioner 20 at an angle that will enable the opening 58 of the diffuser 50 to deliver medical gas at a somewhat downward angle (relative to the top edge 85 of the pillowcase 70) toward the subject's mouth and nose.

In use, the edge 95 of the positioner receptacle 88 may be tucked between a lower surface of the ledge 36 of the positioner and an upper extent of a diffuser 50 that has been coupled to the positioner 20. A positioner receptacle 88 may cover a positioner 20 in a manner that prevents the subject from unintentionally contacting the positioner 20 (e.g., as the individual moves as he or she sleeps or otherwise rests, etc.), which may minimize the possibility of the subject being injured by unintended contact with the positioner 20.

In some embodiments, a medical gas delivery system 10 may include a pillowcase 70 with at least one positioner fastener 76 and/or positioner receptacle 88, at least one positioner 20, and a diffuser 50 that may be coupled to each positioner 20.

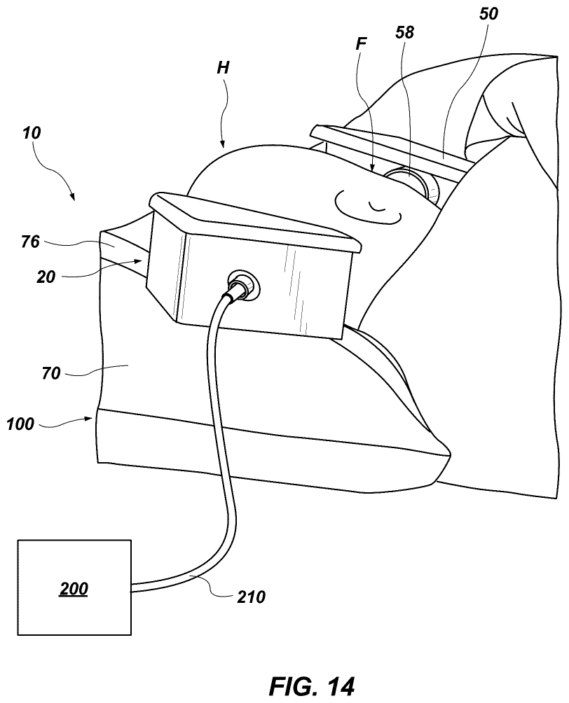

As shown in FIG. 14, a medical gas may be delivered to a subject with such a medical gas delivery system 10 by arranging the positioner 20 on a positioner fastener 76 and, optionally, within a positioner receptacle 88 (FIGS. 12 and 13) of a pillowcase 70 on a pillow 100 where the subject will rest their head H. The positioner 20 is placed on the positioner fastener 76 and, optionally, within the positioner receptacle 88 at a location and at an angle that will enable the opening 58 of a diffuser 50 coupled to the positioner 20 to direct the medical gas from a source 200 to a location over the upper surface 78 of the pillowcase 70 where the subject's head H will rest. In some embodiments, another positioner 20 and diffuser 50 may be arranged relative to another positioner fastener 76 and, optionally, within another positioner receptacle 88 on an opposite side of the pillowcase 70. Such an arrangement may ensure that the subject will receive the medical gas regardless of the orientation of their head H on the pillow 200.

The subject's head H may be placed on the upper surface 78 of the pillowcase 70 and, thus, on the pillow 200 adjacent to the positioner(s) 20. If desired, the position of each positioner may be adjusted (e.g., moved, re-oriented, etc.) to increase the subject's comfort, optimize the efficiency with which the medical gas delivery system delivers the gas to the subject, or the like. With the subject's head positioned on the surface, the medical gas may be delivered to and through the diffuser 50 (FIGS. 11-13) to the face F of the subject.

Maintenance of the medical gas delivery system 10 may include removal and disposal of the diffuser 50 after a predetermined period of time, a predetermined number of uses, etc. The conduit 210 between the source 200 of medical gas and the diffuser 50 should be replaced periodically. In addition, the positioner 20 should be cleaned or sanitized periodically and the pillowcase 70, if any, should be laundered periodically. The amount of time for each of these actions may differ, depending upon exposure of a particular component to the subject. Alternatively, some or all of these actions may be performed on the same schedule (e.g., the pillowcase 70 may be laundered, the diffuser 50 replaced, and the positioner 20 cleaned at the same time, etc., e.g., more frequently than the conduit 210 is replaced).

Although this disclosure and the accompanying drawings provide many specifics, they should not be construed as limiting the scopes of any of the appended claims, but merely as providing information pertinent to some specific embodiments that may fall within the scopes of the appended claims. The scopes of the appended claims may encompass other embodiments. Features from different embodiments may be employed in combination. All additions to, deletions from, and modifications of the disclosed subject matter that fall within the scopes of the claims are to be embraced by the claims.

1. A medical gas delivery system, comprising:
a positioner including:
a positioning wall with a concave front surface defining a recess; and
an aperture through the positioning wall; and
a diffuser including:
a proximal portion shaped to be received by the aperture and defining an inlet of the diffuser through a proximal end of the proximal portion;
an intermediate portion adjacent to a distal side of the proximal portion; and
a distal portion adjacent to a distal side of the intermediate portion with a diameter that exceeds a diameter of the proximal portion, a distal end of the distal portion defining a delivery face of the diffuser.

2. The medical gas delivery system of claim 1, wherein the positioner further includes:
a ledge over the positioning wall and the recess defined by the front surface of the positioning wall.

3. The medical gas delivery system of claim 1,
wherein the positioner further includes:
a support wall behind the positioning wall.

4. The medical gas delivery system of claim 1, wherein the positioner further includes:
a fastener carried by a bottom of the positioning wall.

5. The medical gas delivery system of claim 4, wherein the fastener comprises a touch fastener.

6. The medical gas delivery system of claim 1, wherein the aperture includes a coupler that couples to the proximal portion of the diffuser.

7. The medical gas delivery system of claim 1, further comprising:
a pillowcase including:
an upper surface; and
a positioner fastener on the upper surface.

8. The medical gas delivery system of claim 7, wherein the positioner fastener comprises a touch fastener.

9. The medical gas delivery system of claim 8, wherein the positioner fastener is elongated.

10. The medical gas delivery system of claim 7, wherein the positioner fastener is located on the upper surface of the pillowcase so as to position the delivery face of the diffuser toward a location of face of a subject whose head rests upon the upper surface of the pillowcase.

11. The medical gas delivery system of claim 7, comprising a plurality of positioner fasteners.

12. The medical gas delivery system of claim 7, wherein the pillowcase further includes:
a positioner receptacle on the upper surface, over the positioner fastener.

13. The medical gas delivery system of claim 12, comprising a plurality of positioner receptacles.

14. A method for delivering a medical gas to a subject, comprising:
coupling a proximal portion of a diffuser to a positioning wall of a positioner;
placing the positioner at a first location on an upper surface of a pillowcase on which a head of the subject will be placed so as to place a delivery face of the diffuser adjacent to a second location on the upper surface where the head of the subject will be placed;
orienting the delivery face of the diffuser toward the second location; and
removably securing the positioner to the first location.

15. The method of claim 14, wherein placing the positioner at the first location comprises placing the delivery face of the diffuser toward the second location.

16. The method of claim 14, wherein:
coupling the proximal portion of the diffuser to the positioning wall of the positioner comprises coupling proximal portions of two diffusers to positioning walls of two positioners;
positioning the positioner at the first location comprises positioning the two positioners at two first positions on opposite sides of the upper surface of the pillowcase;
orienting the delivery face of the diffuser comprises orienting delivery faces of two diffusers coupled to the two positioners toward the second location; and
removably securing comprises removably securing the two positioners to the two first positions.

17. The method of claim 14, further comprising:
delivering the medical gas through the delivery face of the diffuser to the face of the subject.

18. The method of claim 14, wherein:
placing the positioner comprises placing the positioner within a positioner receptacle of the pillowcase with the delivery face of the positioner being exposed through a primary opening of the positioner receptacle.

19. A diffuser for medical gas delivery, comprising:
a use indicator that provides a perceptible indication that a useful life of the diffuser has ended.

20. The diffuser of claim 19, wherein:
the use indicator provides the perceptible indication after a predetermined duration of time has elapsed; or
the use indicator provides the perceptible indication after the diffuser has been used a predetermined amount of time.
